(19) United States
(12) Patent Application Publication
(10) Pub. No.: US 2021/0020284 A1
(43) Pub. Date: Jan. 21, 2021

(54) Health-Wellness-Medical Profile Maintenance Based on Content Analysis of Audio/Visual Posts

(71) Applicant: Nano Global Corp., Austin, TX (US)

(72) Inventors: Steven Papermaster, Austin, TX (US); Zoltan Papp, Austin, TX (US); Aaron Papermaster, Austin, TX (US)

(21) Appl. No.: 16/929,977

(22) Filed: Jul. 15, 2020

Related U.S. Application Data
(60) Provisional application No. 62/874,500, filed on Jul. 15, 2019.

(51) Int. Cl. G16H 15/00; G16H 10/60; G16H 80/00; G16H 30/00; G16H 40/60

(52) U.S. Cl. CPC G16H 15/00; G16H 10/60; G16H 40/60; G16H 30/00; G16H 80/00

(57) ABSTRACT

A method that facilitates the maintenance (e.g. creation and update) of health-wellness-medical (HWM) profiles of people and provides a social platform focused on HWM matters for a like-minded community, and provides access to databases of HWM matters, HWM products, HWM coaches, and medical professionals.

### BACKGROUND

The mobile apps exist that allow users to create a short video of themselves, which may feature music in the background, can be sped up, slowed down or edited with a filter, To create a music video with such an app, users can choose background music from a wide variety of music genres, edit with a filter and record a 15-second video with speed adjustments before uploading it to share with others on an associated social platform.

There are also exists many apps and websites that provide medical information. Users can often search for more information on a given condition, search symptoms, or prescription drug information.

### BRIEF DESCRIPTION OF THE DRAWINGS

FIG. 1 illustrates an example audio/visual post interface in accordance with the present disclosure.

FIG. 2 illustrates an example HWM profile interface in accordance with the present disclosure.

FIG. 3 illustrates an example HWM timeline interface in accordance with the present disclosure,

FIG. 4 illustrates a system configured to facilitate health-wellness-medical profile maintenance based on content analysis of audio/visual posts, in accordance with one or more implementations,

The Detailed Description references the accompanying figures. in the figures, the left-most digit(s) of a reference number identifies the figure in which the reference number first appears. The same numbers are used throughout the drawings to reference like features and components,

### DETAILED DESCRIPTION

Technology is described herein that facilitates the maintenance (e.g., creation and update) of health-wellness-medical (HWM) profiles of people. In addition, the technology is described herein provides a social platform focused on HWM matters for a like-minded community, and it provides access to databases of HWM matters, HWM products, HWM coaches, and medical professionals.

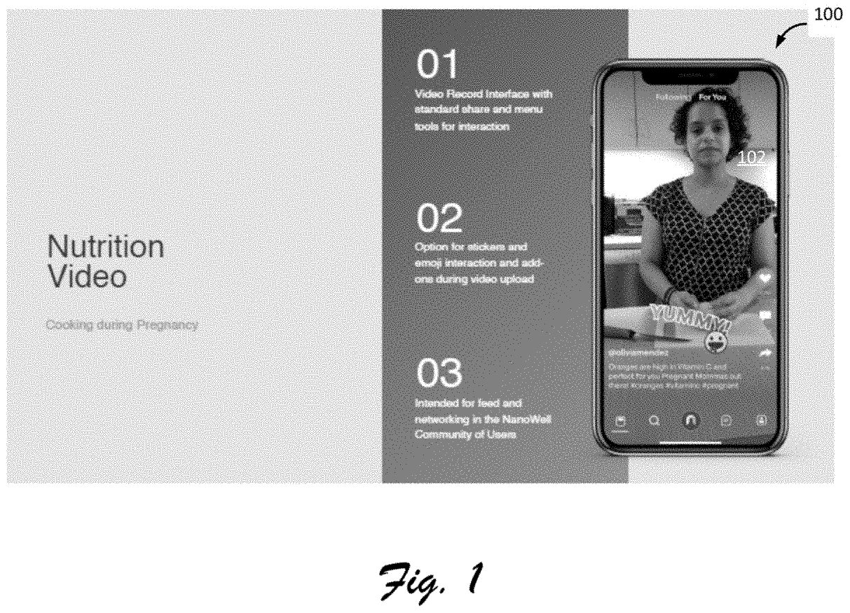

FIG. 1 illustrates a smartphone 100 with a snapshot of an image 102 of an example video post (which is an example of an audio/visual (A/V) post) that a human user posted on a social platform associated with the technology described herein.

In this example, the human poster (who is pictured in the image) uploads or creates the audio/visual (A/V) post. The app and/or social platform obtains the A/V post from the human poster.

A content analyzer analyzes the content of the A/V post. For example, any of the images, text, and audio content of the A/V post may be analyzed. The analysis may include object recognition, optical character recognition, speech recognition, or the like. The functions of the content analyzer may be performed by the related app installed on the user's mobile device alone and/or by a backend/cloud computing resource/service. More particularly, the content analyzer may be accomplished by AI/ML/DL engine discussed in the appendices.

An HWM determiner determines the health-wellness-medical (HWM) information that is contained in the analyzed content. That is, the HWM determines whether such HWM information exists in the analyzed content and whether there is more than one discrete instance of such information. This functionality may also be accomplished by AI/ML/DL engine discussed in the appendices.

For each discrete instance of the determined HWM information in the analyzed content, an HWM identifier identifies the particular type, kind, property, or quality of that information. For example, if Olivia (the human poster of FIG. 1) discusses pregnancy, then the identifier identifies “pregnancy” as the particular HWM information being discussed. This functionality may also be accomplished by AI/ML/DL engine discussed in the appendices.

Then the technology associates the identified HWM information with the human poster and an associated HWM profile for that person is updated accordingly. For example, the HWM profile for Olivia may be updated to reflect that she is pregnant.

In some instances, the technology may determine that the content refers to another human in the context of the HWM information. For example, Olivia may say that her husband is anemic. In that case, the technology determines that the human poster is referring to someone other than themselves. Then it identifies who that is. For example, the technology may look up who Olivia's husband is to determine who she is referred to. Then the technology associates the identified HWM information with the other human (e.g., Olivia's husband) and updates an associated HWM profile for that person accordingly.

In some instances, the technology may be unable to determine who the referenced other human might be. In that case, the technology may inquire to the poster to clear up any ambiguities. If it seems likely that there is no existing HWM profile for the other person (e.g., an ultrasound for an unborn child; of course, the unborn child has no profile yet), then the technology creates a new HWM profile for that person.

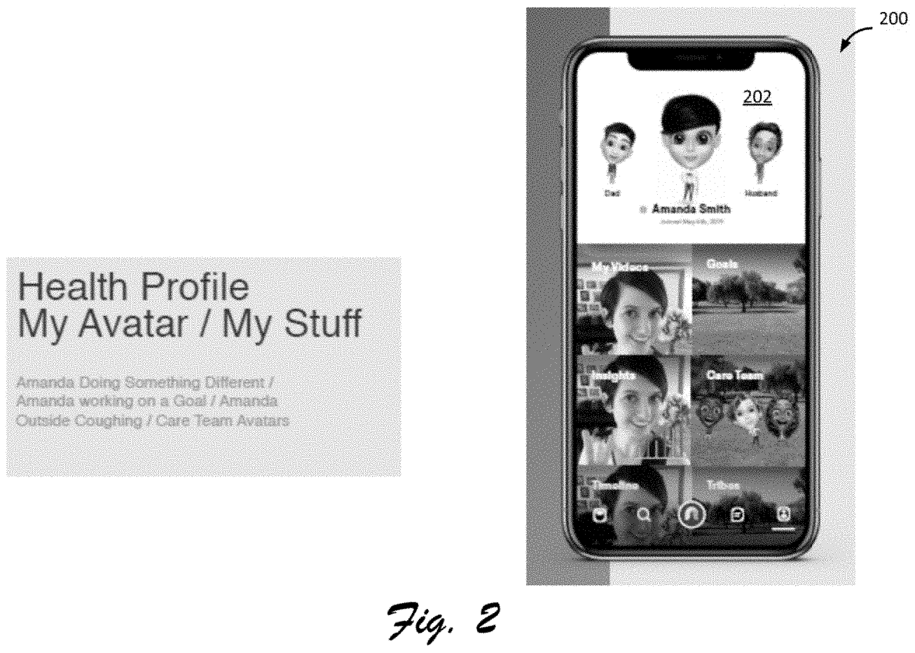

FIG. 2 shows a mobile device 200 showing a screenshot 202 of Amanda's HWM profile, which is called her “Health Profile” in the drawing.

A HWM profile associated with a particular human stores health, wellness, and medical information about that human. Such information includes (but is not limited to;: electronic medical records, protected health information, lab tests results, fitness information, diet information, vital signs, prescriptions, supplements, sleep habits, diseases, dental records, environment factors, genetic records of self and relatives, and the like.

The HWM profiles may be stored in a format that makes the data immutable but amendable with tracking. This is commonly accomplished using a distributed ledger format or a blockchain format.

In addition, the technology described herein may include selecting a service, product, health coach, and/or medical professional that is relevant to the identified HWM information and then sending promotional material regarding that selected product, service, coach, or medical professional to the human poster (and/or to the other human). In addition or alternatively, the technology providing content information or a mechanism to communicate about the selected product, service, coach, or medical professional to the human poster (and/or to the other human).

Furthermore, the technology described herein may include forecasting events for the human poster based on the identified. HWM information and/or their HWM profile and communicating with human poster regarding the forecasted events. For example, the technology may forecast the typical pregnancy timeline of events for Olivia for'her new pregnancy.

Further still, the technology described herein may allow comprising sharing the A/V post with other humans based on their expressed or inferred interest in the identified HWM information.

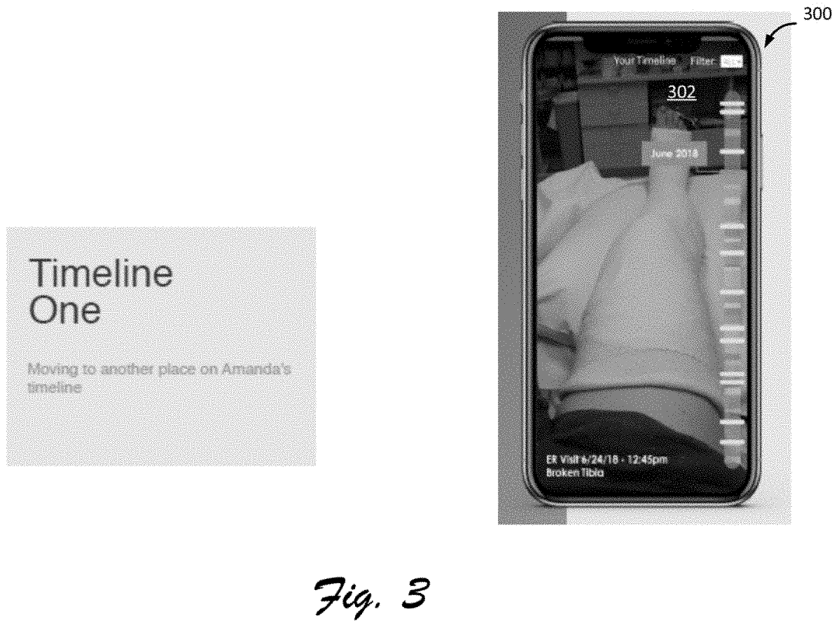

FIG. 3 shows a mobile device 300 showing an image 302 of a point on the timeline of Amanda, Amanda's timeline shows the many HWM events/postings that Amanda has shared via the social platform with friends and other interested parties.

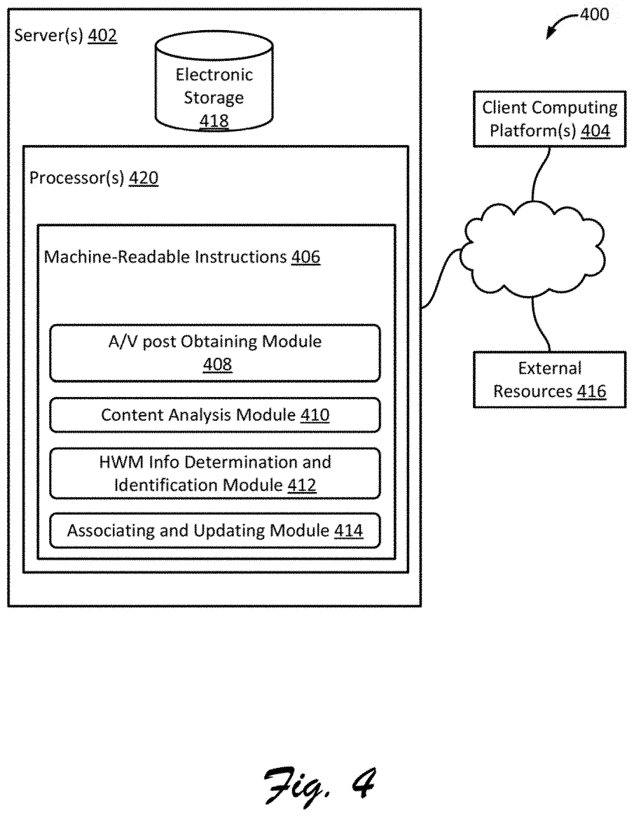

FIG. 4 illustrates a system 400 configured to facilitate amelioration based on detection of biological cells or biological substances, in accordance with one or more implementations. The system 400 is an example of the amelioration system 242 of electronic device 200 and the amelioration subsystem 368 of system 300,

In some implementations, system 400 may include one or more servers 402. Server(s) 402 may be configured to communicate with one or more client computing platforms 404 according to a client/server architecture and/or other architectures. Client computing platform(s) 404 may be configured to communicate with other client computing platforms via server(s) 402 and/or according to a peer-to-peer architecture and/or other architectures. Users may access system 400 via client computing platform(s) 404.

Server(s) 402 may be configured by machine-readable instructions 406, Machine-readable instructions 406 may include one or more instruction modules. The instruction modules may include computer program modules. The instruction modules may include one or more of A/V post obtaining module 408, content analysis module 410, HWM info detection and identification module 412, associating and updating module 414, and/or other instruction modules.

A/V post obtaining module 408 may be configured to obtain an audio/visual (A/V) post created by a user (i.e., human poster). An app and/or social platform obtains the A/V post from the human poster.

The content analysis module 410 is configured to analyze the content of the A/V post. For example, any of the images, text, and audio content of the A/V post may be analyzed, The analysis may include object recognition, optical character recognition, speech recognition, or the like. The functions of the content analyzer may be performed by the related app installed on the user's mobile device alone and/or by a backend/cloud computing resource/service. More particularly, the content analyzer may be accomplished by AI/ML/DL engine discussed in the appendices.

The HWM info detection and identification module 412 is configured to determine the health-wellness-medical (HWM) information that is contained in the analyzed content. That is, the HWM determines whether such HWM information exists in the analyzed content and whether there is more than one discrete instance of such information. This functionality may also be accomplished by AI/ML/DL engine discussed in the appendices.

(0030) in addition, the HWM info detection and identification module 412 is configured to, for each discrete instance of the determined HWM information in the analyzed content., identifies the particular type, kind, property or quality of that information. For example, if Olivia (the human poster of FIG. 1) discusses pregnancy, then the identifier identifies “pregnancy” as the particular HWM information being discussed. This functionality may also be accomplished by AI/ML/DL engine discussed in the appendices.

The associating and updating module 414 is configured to associate the identified HWM information with the human poster and update an associated HWM profile for that person accordingly. For example, the HWM profile for Olivia may be updated to reflect that she is pregnant.

In some instances, the technology may determine that the content refers to another human in the context of the HWM information. For example, Olivia may say that her husband is anemic, In that case, the technology determines that the human poster is referring to someone other than themselves. Then it identities who that is. For example, the technology may look up who Olivia's husband is to determine who she is referred to. Then the technology associates the identified HWM information with the other human (e.g., Olivia's husband) and updates an associated HWM profile for that person accordingly.

In some instances, the technology may be unable to determine who the referenced other human might be. In that case, the technology may inquire to the poster to clear up any ambiguities. If it seems likely that there is no existing HWM profile for the other person (e.g. an ultrasound for an unborn child; of course, the unborn child has no profile yet), then the technology creates a new HWM profile for that person,

In some implementations, server(s) 402, client computing platform(s) 404, and/or external resources 416 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which server(s) 402, client computing platform(s) 404, and/or external resources 416 may be operatively linked via some other communication media.

A given client computing platform 404 may include one or More processors configured to execute computer program modules. The computer program modules may be configured to enable an'expert or user associated with the given client computing platform 40 to interface with system 400 and/or external resources 416, and/or provide other functionality attributed herein to client computing platfonn(s) 404. By way of non-limiting example, the given client computing platform 404 may include one or more of a desktop computer, a laptop computer, a handheld computer, a tablet computing platform, a NetBook, a Smartphone, a gaming console, and/or other computing platforms.

External resources 416 may include sources of information outside of system 400, external entities participating with system 400, and/or other resources. in some implementations, some or all of the functionality attributed herein to external resources 416 may be provided by resources included in system 400.

Server(s) 402 may include electronic storage 418, one or more processors 420, and/or other components. Server(s) 402 may include communication lines, or ports to enable the exchange of information with a network and/or other computing platforms. Illustration of servens) 402 in FIG. 4 is not intended to be limiting. Server(s) 402 may include a plurality of hardware, software, and/or firmware components operating together to provide the functionality attributed herein to server(s) 402. For example, server(s) 402 may be implemented by a cloud of computing platforms operating together as server(s) 402.

Electronic storage 418 may comprise non-transitory storage media that electronically stores information. The electronic storage media of electronic storage 418 may include one or both of system storage that is provided integrally (i.e., substantially non-removable) with server(s) 402 and/or removable storage that is removably connectable to servers) 402 via, for example, a port (e.g, a USB port, a firewire port, etc.) or a drive (e.g., a disk drive, etc.). Electronic storage 418 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 418 may include one or more virtual storage resources (e.g., cloud storage, a virtual private network, and/or other virtual storage resources). Electronic storage 418 may store software algorithms, information determined by processor(s) 420, information received from server(s) 402, information received from client computing platform(s) 404, and/or other information that enables server(s) 402 to function as described herein.

Processor(s) 420 may be configured to provide information processing capabilities in server(s) 402. As such, processor(s) 420 may include one or more of a digital processor, an analog processor, a digital circuit designed to process information, an analog circuit designed to process information, a state machine, and/or other mechanisms for electronically, processing information. Although processor(s) 420 is shown in FIG. 4 as a single entity, this is for illustrative purposes only. In some implementations, processor(s) 420 may include a plurality of processing units. These processing units may be physically located within the same device, or processor(s) 420 may represent processing functionality of a plurality of devices operating in coordination. Processor(s) 420 may be configured to execute modules 408, 410, 412, and/or 414, and/or other modules. Processor(s) 420 may be configured to execute modules 408, 410, 412, and/or 414, and/or other modules by software; hardware; firmware; some, combination of software, hardware, and/or firmware; and/or other mechanisms for configuring processing capabilities on processor(s) 420. As used herein, the term “module” may refer to any component or set of components that perform the functionality attributed to the module. This may include one or more physical processors during execution of processor readable instructions, the processor readable instructions, circuitry, hardware, storage media, or any other components.

It should be appreciated that although modules 408, 410, 412. and/or 414 are illustrated in FIG. 4 as being implemented within a single processing unit, in implementations in which processor(s) 420 includes multiple processing units, one or more of modules 408, 410, 412, and/or 414 may be implemented remotely from the other modules. The description of the functionality provided by the different modules 408, 410, 412, and/or 414 described below is for illustrative purposes, and is not intended to be limiting, as any of modules 408, 410, 412, and/or 414 may provide more or less functionality than is described. For example, one or more of modules 408, 410, 412. and/or 414 may be eliminated, and some or all of its functionality may be provided by other ones of modules 408, 410, 412, and/or 414. As another example, processor(s) 420 may be configured to execute one or more additional modules that may perform some or all of the functionality attributed below to one of modules 408, 410, 412, and/or 414.

### ADDITIONAL AND ALTERNATIVE IMPLEMENTATION NOTES

In the above description of example implementations, for purposes of explanation, specific numbers, materials, configurations, and other details are set forth in order to better explain the present disclosure. However, it will be apparent to one skilled in the art that the subject matter of the claims may be practiced using different details than the examples ones described herein. In other instances, well-known features are omitted or simplified to clarify the description of the example implementations.

The terms “techniques” or “technologies” may refer to one, or more devices, apparatuses, systems, methods, articles of manufacture, and/or executable instructions as indicated by the context described herein

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

These processes are illustrated as a collection of blocks in a logical flow graph, which represents a sequence of operations that may be implemented in mechanics alone, with hardware, and/or with hardware in combination with firmware or software. In the context of software/firmware, the blocks represent instructions stored on one or more non-transitory computer-readable storage media that, when executed by one or more processors or controllers, perform the recited operations.

Note that the order in which the processes are described is not intended to be construed as a limitation, and any number of the described process blocks can be combined in any order to implement the processes or an alternate process. Additionally, individual blocks may be deleted from the processes without departing from the spirit and scope of the subject matter described herein.

The term “computer-readable media” is non-transitory computer-storage media or non-transitory computer-readable storage media. For example, computer-storage media or computer-readable storage media may include, but are not limited to, magnetic storage devices (e.g., hard disk, floppy disk, and magnetic strips), optical disks (e,g., compact disk (CD) and digital versatile disk (DVD)), smart cards, flash memory devices (e.g. thumb drive, stick, key drive, and SD cards), and volatile and non-volatile memory (e.g., random access memory (RAM), read-only memory (ROM)).

What is claimed is:

1. A method that facilitates maintenance (e,g., creation and update) of health-wellness-medical (HWM) profiles, the method comprising:
obtaining an audio/visual (A/V) post created by a human poster;
analyzing content of the A/V post;
determining that health-wellness-medical (HWM) information is contained in the analyzed content;
identifying the determined HWM information in the analyzed content;
associating the identified HWM information with the human poster;
updating an HWM profile of that human poster with the associated HWM information.

2. A method of claim 1 further comprising:
determining that the HWM information is associated with a human other than the human poster;
identifying that other human;
associating the identified HWM information with the other human;
updating an HWM profile of that other human with the associated HWM information.

3. A method of claim 1 further comprising:
determining that the HWM information is associated with a human other than the human poster;
associating the identified HWM information with the other human;
determining that no HWM profile exists for that other human;
in response to the &termination that no HWM profile exists, creating an HWM profile for that other human;
updating an HWM profile of that other human with the associated HWM information,

4. A method of claim 1, wherein the HWM profiles are stored in a distributed ledger,

5. A method of claim 1, wherein HWM profiles are stored in a blockchain format.

6. A method of claim 1, wherein fIWM profiles in an immutable but amendable format.

7. A method of claim 1, wherein an HWM profile associated with human stores health, wellness, and medical information about that human.

8. A method of claim 1, wherein an HWM profile associated with human stores health, wellness, and medical information about that human from one or more various sources from a group consisting of:
wearable device;
smart device:
digital medical records;
genetic records of that human and/or related family members; and,
environmental sensor data gathered from sensors located in the environment in which the human lives (e.g, home, work, etc).

9. A method of claim 1, wherein the HWM profile includes information collected and analyzed as part of the sci-data of the appendix entitled “FACILITATION OF THE DISTRIBUTION OF SCIENTIFIC DATA.”

10. A method of claim I, further comprising:
selecting a product relevant to the identified HWM information;
sending promotional material for that selected product to the human poster.

11. A method of claim 1, further comprising:
selecting a service relevant to the identified HWM information;
sending promotional material for that selected service to the human poster.

12. A method of claim 1, further comprising:
selecting a health coach with expertise that is relevant to the identified HWM information:
sending promotional material for that selected health coach to the. human poster OR providing contact information for that selected health coach to the human poster OR provide a mechanism for that selected health coach to contact the human poster.

13. A method of claim 1, farther comprising:
selecting a medical professional with expertise that is relevant to the identified HWM information:
sending promotional material for that selected medical professional to the human poster OR providing contact information for that selected medical professional to the human poster OR provide a mechanism for that selected medical professional to contact the human poster.

14. A method of claims 10-13 further comprising receiving compensation from the product seller/service seller/health coach/medical professional for products/services sold via the actions of claims 10-13.

15. A method of claim 1 further comprising forecasting events for the human poster based on the identified HWM information and/or their HWM profile,

16. A method of claim 1 further comprising:
forecasting events for the human poster based on the identified HWM information and/or their HWM
communicating with human poster regarding the forecasted events.

17. A method of claim 1 further comprising sharing the A/V post with other humans based on their expressed or inferred interest in the identified HWM information.
